(19) United States
(12) Patent Application Publication
(10) Pub. No.: US 2004/0226080 A1
(43) Pub. Date: Nov. 18, 2004

(54) Detachable diving goggle

(75) Inventor: Chih-Cheng Shiue, Rancho Santa Fe, CA (US)

Correspondence Address: BRUCE H. TROXELL, SUITE 1404, 5205 LEESBURG PIKE, FALLS CHURCH, VA, 22041, US

(73) Assignee: QDS Injection Molding LLC.

(21) Appl. No.: 10/640,041

(22) Filed: Aug. 14, 2003

(30) Foreign Application Priority Data
May 16, 2003 (TW) 092208964

(51) Int. Cl. A61F 9/02

(52) U.S. Cl. 2/426

(57) ABSTRACT

The present invention is relating to a detachable diving goggle including a lens, a frame, a band, and a soft skirt. The lens and the frame are made integrally. The frame has the groove and the band is assembled at the two sides of the frame; The soft skirt has a flange that fits into the groove of the frame. Since the flange and the groove can either be used to attach or separate the skirt and the frame, the diving goggle can transform into a pair of sun shielding lens.

### FIELD OF THE INVENTION

The present invention is a type of detachable diving goggle, which can be used during the following activities: diving, scuba diving, and swimming (the term “water sport” will now be used in place of the previously listed activities). On the other hand, the soft skirt of the goggle can be easily unassembled to become a pair of sun shielding lens when one engages in non-water sports activities.

### BACKGROUND OF THE INVENTION

Diving goggle that can cover the user's eyes or nose is the basic equipment for many water recreational sports. The goal is to prevent water from going into the user's eyes, which makes the user uncomfortable.

People usually need a pair of diving goggles while engaging in water sports. However, the user usually needs to take off the goggles and put on a pair of sun shielding lenses while resting. The reason is that the air pressure and the weight of diving goggles is heavier, also, due to the temperature change from water to land, fog can be formed on the goggles and interrupt the user's vision. Therefore, in general, diving goggles are not usually worn for a long time while resting.

A pair of diving goggles usually includes several basic parts such as the lenses, the soft skirt and the frame. The frame part is either comprised of simply one main frame, or in some cases, divided into a main frame and a sub-frame. The structure of diving goggles usually has the lenses and the skirt held together by the main frame and the sub-frame. However, in structure, there is also providing the lenses, skirt and the frame conjugate in one body.

From the structures described above, it is hard to achieve the goal of producing a pair of goggles that can both function as diving goggles for water sports, and as sun shielding lens.

### SUMMARY OF THE INVENTION

A main purpose of the present invention is providing diving goggles with the extra function of sun shielding.

The distinctiveness of the diving goggle of the present invention is the ease of which the diving goggle can be converted into sun blocking lens.

The diving goggle described above has not yet been found in the market. The present invention allows the frame and the skirt to assemble and disassemble. This kind of structure can also allow the lenses and the frame to be manufactured in one piece.

### BRIEF DESCRIPTION OF THE DRAWINGS

FIG. 1: the perspective view of the present invention.

FIG. 2: the exploded view of the present invention.

FIG. 3: the view showing the embodiment (including the skirt) of the present invention.

FIG. 4: the view showing the embodiment (not including the skirt) of the present invention.

FIG. 5: the cross section view of the skirt and the users eye location of the present invention.

FIG. 6: the perspective view showing the second embodiment of the present invention.

### DETAILED DESCRIPTION OF PREFERRED EMBODIMENTS

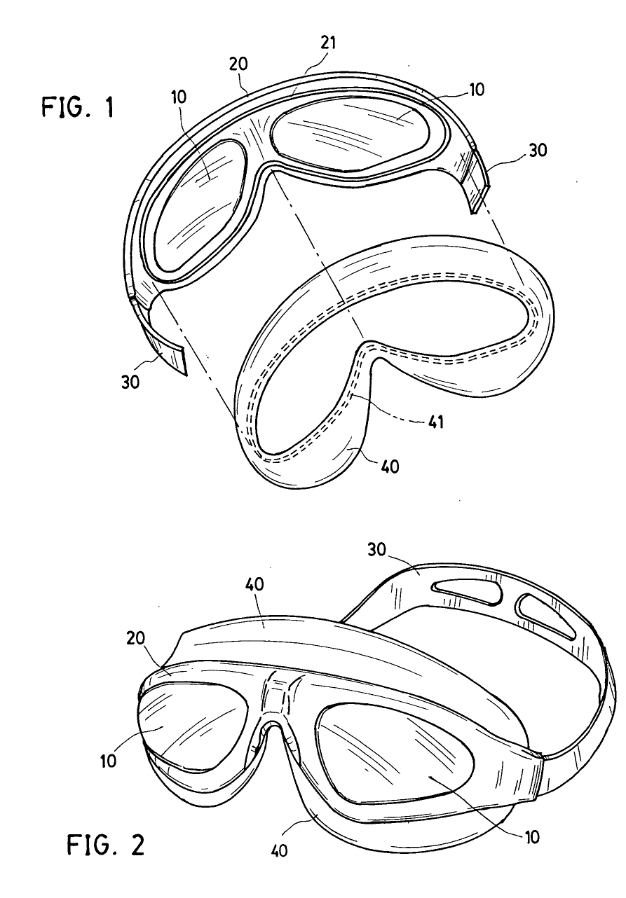

According to FIG. 1 and FIG. 2, the structure of the present invention includes at least lenses 10, a frame 20, a band 30 and a skirt 40.

Lenses 10 are made by flat and transparent material, it is conjugate and made in integrally with the frame 20. The band 30 is located on the two sides of the frame 20.

Groove 21 is assembled on the inner side of the frame 20.

The skirt 40 is made by soft material (such as silicon) for used to cover the surrounding of the eyes and nose. A flange41 is assembled on the skirt 40, which can fit into the groove 21, so that the skirt 40 and the frame 20 can be assembled together. By this way water can be prevented from entering the goggle

Flange41 is also made by soft material such as silicon with certain flexibility. By assembled into the groove 21, the goggle can be waterproof.

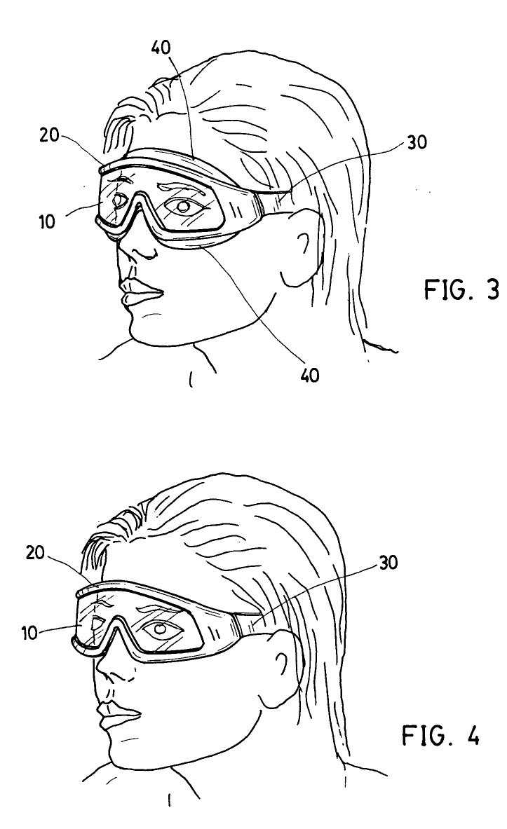

According to FIG. 3 and FIG. 4, the embodiment of the present invention, we can find that this functioning and stylish design can surely be a useful product.

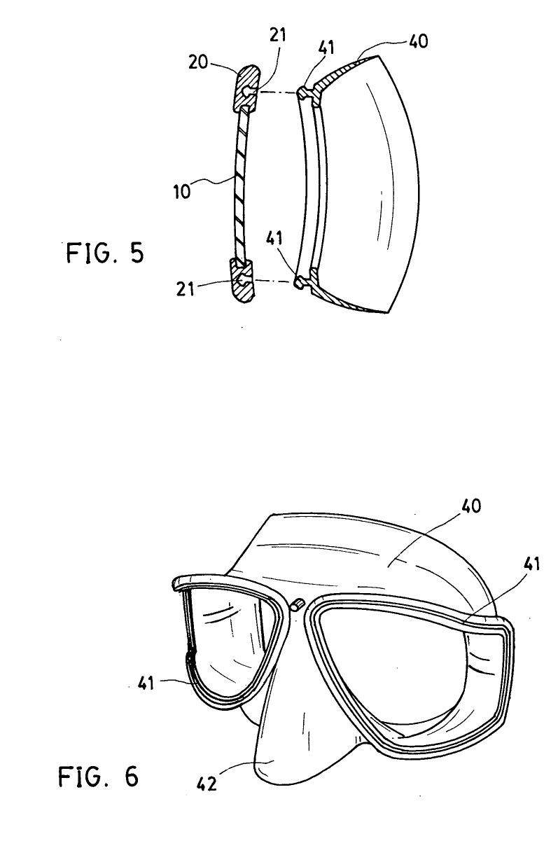

According to FIG. 5, the flange41 of the skirt 40 can fit into the groove 21 of the frame 20. This kind of structure is not limited to what is shown on the figure, as long as the frame 20 and the skirt 40 can be assembled together and make the goggle waterproof; they should belong to the patent of this present invention.

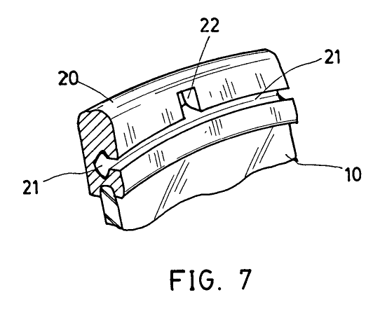

According to FIG. 6, another embodiment of the skirt 40, the skirt 40 includes the nose cover 42, which means the skirt 40 is not limited to only covering the eyes.

Moreover, several notch 22 (which was not drawn) can be drilled above the frame 20, where several protruding tenons 43 on the skirt 40 are assembled to the notches 22. By this way, the skirt 40 and the frame 20 can be easily assembled to each other. This can also be achieved by pressing flange 41 into the groove 21. During operation, the external pressure to the skirt 40 can apply on the protruding tenons 43 and the notches 22, which can decrease the amount of the pressure to the flange 41 and the groove 21, so that the possibility of water entering the goggle can be minimized.

When the user does not need the skirt 40, pulling flange41 away from the groove 21 from the corner of the skirt 40 can pull off the skirt 40. By doing this, the diving goggle can become a pair of sun shielding lens.

According to FIG. 7, the embodiment shows that the frame 20, the gutter21, and the notches 22. The amount of notches 22 is not limit to only one, it can also connect with the groove 21, so that the flange 40 and the protruding tenons 43 can connect each other (not drawn).

The position of the flange and the groove can also be opposite to each other, such as putting the flange on the frame and putting the groove on the skirt. By the way, the frame and the skirt remain assembled to each other this way.

When the skirt 40 and the frame 20 are assembled together, a diving goggle can be formed; when the two parts are separated, a pair of sun shielding lens can be formed.

The specialty of the present is that the frame 20 and the skirt 40 can be manually attach to or separate from each other. Also, the attaching method does not limit to groove 21 and flange 41.

What is claimed is:

1. A detachable diving goggle, which includes:
At least a lens, which attachable to the frame;
A frame: with the structure to allow the frame and the skirt attach each other, and the band is located on the two sides of the frame;
A skirt: includes the structure allows itself to attach with the frame;
Whereby, by attaching and separating the skirt and the frame to form either a diving goggle or sun shielding lens.

2. The detachable diving goggle as claimed in claim 1, the groove is located on the frame and the flange is located on the skirt, whereby the groove and the flange can fit into each other.

3. The detachable diving goggle as claimed in claim 1, wherein the notch is located on the frame and the protruding tenon is located on the skirt, whereby the notch and the protruding tenon can fit into each other.

4. The detachable goggle as claimed in claim 1, wherein the flange is located on the frame and groove is located on the skirt, whereby the flange and the gutter can fit into each other.

5. The detachable goggle as claimed in claim 1, wherein the protruding tenon is located on the frame and notch is located on the skirt, whereby the protruding tenon and the notch can fit into each other.

6. A detachable diving goggle, which comprising:
A frame having at least a lens and a band connected to both side thereof, a detached member is provided on said frame;
A skirt having the detachable member respective to said detached member.

7. A detachable diving goggle as claimed in claim 6, wherein said detached member is groove and said detachable member is tenon.

8. A detachable diving goggle as claimed in claim 6, wherein said detached member is tenon and said detachable member is groove.
